(19) United States
(12) Patent Application Publication
(10) Pub. No.: US 2026/0179767 A1
(43) Pub. Date: Jun. 25, 2026

(54) SYSTEM AND METHOD FOR MANAGING TREATMENT OF BEHAVIORAL HEALTH CONDITIONS

(71) Applicants: Roderick L Brown, Southfield, MI (US); Dwayne E Goldsmith, Northville, MI (US); Vassal N Johnson, Southfield, MI (US)

(72) Inventors: Roderick L Brown, Southfield, MI (US); Dwayne E Goldsmith, Northville, MI (US); Vassal N Johnson, Southfield, MI (US)

(73) Assignee: ASENDIO, Inc, Ann Arbor, MI (US)

(21) Appl. No.: 19/434,228

(22) Filed: Dec. 29, 2025

Related U.S. Application Data
(60) Provisional application No. 63/738,665, filed on Dec. 24, 2024.

(51) Int. Cl. G16H 40/67; G16H 10/20; G16H 20/70

(52) U.S. Cl. CPC G16H 40/67; G16H 10/20; G16H 20/70

(57) ABSTRACT

The present invention discloses a system for managing treatment of behavioral health conditions including substance use disorder (SUD). The system comprises a computing device in communication with a database and user devices via a network. The computing device provides a questionnaire comprising a plurality of categories. Each category comprises a set of questions to assess progress of the client. The computing device enables a clinician to input a score for each question based on analyzing response of client to each question. The computing device is configured to apply predefined scoring guidelines for the input score and generate the score data including the category score data for each category and the recovery score data for the questionnaire. The computing device enables the clinician to continuously evaluate and track the progress of the client, and provide a treatment plan based on current score data and progress data of the client.

### CROSS REFERENCE TO RELATED APPLICATION

This application claims the priority benefit of the U.S. Patent Application No. 63/738,665 titled “SYSTEM AND METHOD FOR TREATING SUBSTANCE USE DISORDER”, filed on Dec. 24, 2024, the contents of which are hereby incorporated by reference in their entirety.

### TECHNICAL FIELD

The present invention generally relates to behavioral health conditions. More particularly, the invention is related to a system and method for managing the treatment of behavioral health conditions including substance use disorder (SUD).

### BACKGROUND

Substance Use Disorder (SUD), is a complex medical condition in which an individual has a diminished ability to control the use of one or more legal or illegal substances. Further, the substances could be alcohol, drugs, or prescription medications. The SUD further involves a range of behavioral, cognitive, and physiological issues including cravings, inability to control substance use, and withdrawal symptoms. Further, repeated usage of substances could affect the brain function system.

SUD could vary in severity from mild to severe with mental health disorders, for example, anxiety or depression. The disorder could impact people and lead to significant personal, social, and economic consequences. Further, the tools are used to monitor the SUD and provide the required support to manage the potential risk. Further, monitoring helps to detect early signs and enables healthcare providers to adjust treatment plans for SUD individuals. Further, a few patent references related to monitoring substance use disorder (SUD) are discussed as follows.

US20210065909 of Keri Donaldson entitled “Method and systems for evaluation of risk of a substance use disorders” discloses a method and system for evaluation of risk of substance use disorders. The system comprises one or more single nucleotide polymorphism (SNP) analyzer, a processor, and a machine-readable storage. The single nucleotide polymorphism (SNP) analyzer is configured to analyze the sample from the patient and provide a first plurality of SNP profiles of each set of specified allelic variants in the sample. The machine-readable storage is configured to store a trained ensemble model. The processor is configured to provide the trained ensemble model with the first plurality of SNP profiles to generate a score indicative of risk to the substance use disorder (SUD) for the patient based on the first plurality of SNP profiles. The system indicates a high risk of developing the substance use disorder when the score exceeds a predefined threshold. Further, the system indicates a lower risk to develop the substance use disorder when the score is less than or equal to the pre-determined threshold.

US20230008561 of Thomas Stevens entitled “Software platform and integrated applications for alcohol use disorder (AUD), substance use disorder (SUD), and other related disorders, supporting ongoing recovery emphasizing relapse detection, prevention, and intervention” discloses a medical device automatically monitoring relapse condition of an AUD/SUD user and automatically applying relapse prevention techniques without human oversight. The device comprises a platform configured to obtain a first set of rules for weighting scores of indications of relapse based on timed events and the sequence of those events. The platform retrieves scores for a set of indications through a platform-connected personal mobile device, which is maintained in close proximity to a user diagnosed with substance use disorder (SUD) and/or Alcohol Use Disorder (AUD). The set of indications include a facial stress tension score, a self-assessment score, a movement score, and a physiology score. The platform determines an intermediate relapse score by weighting and combining the indication scores according to the first set of rules. Subsequently, the platform obtains a second set of rules comprising historical weights of past relapse scores and transitional parameters associated with the user and/or similarly situated individuals under similar conditions. The relapse scores and transitional parameters directly attributable to the user are weighted higher than scores and parameters of similarly situated individuals which themselves are ranked and weighted higher or lower depending on the degree of similarity to the user. The second set of rules is then applied to the intermediate relapse score to calculate a final relapse score.

However, the current system solely relies on genetic data and doesn't consider other important factors including environmental, psychological, treatment consistency and social influences, which might limit the ability to provide a comprehensive assessment of SUD risk. The system further lacks to provide comprehensive data, which could complicate the understanding of a client's progress and treatment requirements. Further, the system also lacks the capability to provide real-time alerts for missed appointments, behavioral irregularities, or medication non-adherence of SUD individuals. Further, the system fails to consistently monitor the health of SUD individuals across different counselors. The lack of consistency in monitoring could negatively affect the adjustment of individualized treatment plans for treating SUD.

Therefore, there is a need for a system and method for managing the treatment of behavioral health conditions including substance use disorder (SUD). Further, the system needs to monitor and track the SUD individual's health. Further, the system needs to provide a comprehensive assessment of SUD risk by considering all the important factors including environmental, psychological, treatment consistency, and social influences. Further, the system needs to track the SUD individual's health to ensure the treatment plans, and identify early signs of relapse. Further, the system needs to provide both numerical data and description data that help in understanding a client's progress and treatment requirements. Additionally, the system needs to provide consistency in monitoring that helps to adjust the treatment plans for treating SUD individual.

### SUMMARY

The present invention discloses a system for managing treatment of behavioral health conditions including mental health conditions and substance use disorder. The system comprises at least one computing device, at least one database in communication with the computing device. The system further comprises one or more user devices including at least one first user device associated with a client or patient, at least one second user device associated with a clinician, and at least one third user device associated with a representative of an organization. The user devices and the database are in communication with the computing device via a network. The database comprises a client data, a clinician data, a score data including a category score data and a recovery score data, and a progress data including the score data of each client assessment.

The computing device comprises one or more artificial intelligence (AI) modules. The computing device comprises at least one memory configured to store a set of program modules and at least one processor configured to execute one or more program modules. The modules comprise a query module, a scoring module, a dashboard module, a reporting module, a clinician module, a client module, a treatment optimization module, a communication module, and a data collection module.

The query module is configured to provide a questionnaire comprising the plurality of categories. Each category comprises a set of questions to assess progress of the client. The query module is further configured to enable the clinician to input score for each question of each category based on analyzing response of client to each question. In one embodiment, the category comprises housing stability category, dosing consistency category, illicit substances category, counseling consistency category, physical and mental health category, legal issues impact category, medication management category, interpersonal relationships category and renew gap category.

The scoring module is configured to apply predefined scoring guidelines for the input score and generate the score data including the category score data for each category and the recovery score data for the questionnaire. The scoring module is configured to assign weighted scores to each question based on a relative impact of each question on recovery success of the client to generate the score data. The data collection module is configured to connect with one or more wearable devices associated with patient and collect biometric data of the patient. The biometric data is mapped to the questionnaire to enable real-time recalibration of the score data and AI-driven decision support.

The dashboard module is configured to provide a dashboard comprising the score data, and a graphical representation of score data for the individual clients and for a group of clients. The dashboard module is further configured to provide a graphical representation of progress data for individual clients and for the group of clients. The reporting module enables to assess performance of different organization by assessing recovery of group of clients of different organization using the score data.

The clinician module is configured to enable the clinician to continuously evaluate and track the progress of the client in treatment. The clinicians evaluate and track by using the score data and the questionnaire for the regular period of time until the recovery score data reaches a predefined score. The clinician module is further configured to enable the clinician to update the score data for each category of questions after each client assessment. The clinician module is configured to enable the clinician to provide the treatment plan based on current score data and progress data of the client.

The client module is configured to enable the client to access the treatment plan provided by the clinician. The treatment optimization module is configured to analyze the progress data, and identify trends and provide optimized treatment recommendations to update the treatment plan based on score data and progress data of the client. The communication module is configured to share the progress data of clients across clinicians and organizations.

The computing device is further in communication with one or more external systems including, but not limited to, Electronic Health Record (EHR) system and Uniform Data System, to at least one of receive and access toxicology screen results to predict SUD mortality risk and to generate score data. The computing device is further configured to facilitate relapse detection, relapse monitoring, and emotional monitoring by analyzing the score data, client responses, and historical progress data to identify patterns indicative of relapse risk and emotional distress. The computing device integrates with existing electronic health records (EHR) systems, such as Methisoft, through secure APIs, to ensure real-time synchronization of client data across multiple platforms.

The computing device is configured to maintain real-time consistency of scoring indices, clinical documentation, and treatment planning across multi-agency, multi-county behavioral health networks. The computing device is further configured to generate personalized recovery trajectories by analyzing temporal patterns in a multi-category scoring index and adjusting clinical plans through an AI-enhanced feedback engine. The computing device is configured to provide role-specific interfaces to deliver AI-guided documentation prompts, compliance tracking, and personalized analytics tailored to counselors, medical staff, and administrative users.

In one embodiment, a method for managing treatment of behavioral health conditions including mental health conditions and substance use disorder is disclosed. The method is executed by the system comprising one or more user devices, a database and at least one computing device in communication with at least one database and the user devices via the network. The user devices including at least one first user device associated with the client, at least one second user device associated with the clinician, and at least one third user device associated with the representative of an organization. The database comprises the client data, the clinician data, the score data including category score data and recovery score data, and the progress data includes score data of each client assessment.

The computing device comprises one or more artificial intelligence (AI) modules. The computing device comprises at least one memory configured to store the set of program modules and at least one processor configured to execute one or more program modules. The modules comprise the query module, the scoring module, the dashboard module, the reporting module, the clinician module, the client module, the treatment optimization module, the communication module and the data collection module.

At one step, the query module, at the computing device, is configured to provide the questionnaire comprising the plurality of categories. Each category comprises the set of questions to assess progress of the client. The category comprises housing stability category, dosing consistency category, illicit substances category, counseling consistency category, physical and mental health category, legal issues impact category, medication management category, interpersonal relationships category and renew gap category. The query module is further configured to enable the clinician to input the score for each question of each category based on analyzing response of client to each question.

At yet another step, the scoring module, at the computing device, is configured to apply predefined scoring guidelines for the input score and generate the score data including the category score data for each category and the recovery score data for the questionnaire. At yet another embodiment, the scoring module, at the computing device, is configured to assigns weighted scores to each question based on the relative impact of each question on recovery success of the client to generate the score data.

At yet another step, the data collection module, at the computing device, is configured to connect with one or more wearable devices associated with patient and collect biometric data of the patient. The biometric data is mapped to the questionnaire to enable real-time recalibration of the score data and AI-driven decision support.

At yet another step, the dashboard module, at the computing device, is configured to provide the dashboard comprising score data, and the graphical representation of score data for individual clients and for the group of clients. The dashboard module is further configured to provide the graphical representation of progress data for individual clients and for the group of clients.

At yet another step, the reporting module, at the computing device, enables to assess performance of different organization by assessing recovery of group of clients of different organization using the score data.

At yet another step, the clinician module, at the computing device, is configured to enable the clinician to continuously evaluate and track the progress of the client in treatment by using the score data and the questionnaire for the regular period of time until the recovery score reaches the predefined score, and enable the clinician to update the score for each category of questions after each client assessment.

At yet another step, the clinician module, at the computing device, is configured to enable the clinician to provide the treatment plan based on current score data and progress data of the client. At yet another step, the client module, at the computing device, is configured to enable the client to access the treatment plan provided by the clinician.

At yet another step, the treatment optimization module, at the computing device, is configured to analyze the progress data, identify trends, and provide optimized treatment recommendations to update the treatment plan based on score data and progress data of the client. At yet another step, the communication module, at the computing device, is configured to share the progress data of clients across clinicians and organizations. The computing device is further configured to facilitate relapse detection, relapse monitoring, and emotional monitoring by analyzing the score data, client responses, and historical progress data to identify patterns indicative of relapse risk and emotional distress.

At yet another step, the computing device is configured to maintain real-time consistency of scoring indices, clinical documentation, and treatment planning across multi-agency, multi-county behavioral health networks. At yet another step, the computing device is configured to generate personalized recovery trajectories by analyzing temporal patterns in a multi-category scoring index and adjusting clinical plans through an AI-enhanced feedback engine. At yet another step, the computing device is configured to provide role-specific interfaces to deliver AI-guided documentation prompts, compliance tracking, and personalized analytics tailored to counselors, medical staff, and administrative users.

Other objects, features and advantages of the present innovation will become apparent from the following detailed description. It should be understood, however, that the detailed description and the specific examples, while indicating specific embodiments of the innovation, are given by way of illustration only, since various changes and modifications within the spirit and scope of the innovation will become apparent to those skilled in the art from this detailed description.

### BRIEF DESCRIPTION OF THE DRAWINGS

The foregoing summary, as well as the following detailed description of the innovation, is better understood when read in conjunction with the appended drawings. For the purpose of illustrating the innovation, exemplary constructions of the innovation are shown in the drawings. However, the innovation is not limited to the specific methods and structures disclosed herein. The description of a method step or a structure referenced by a numeral in a drawing is applicable to the description of that method step or structure shown by that same numeral in any subsequent drawing herein.

FIG. 1 exemplarily illustrates an environment of a system and method for managing treatment of behavioral health conditions, according to an embodiment of the present invention.

FIG. 2 exemplarily illustrates a block diagram of the computing device, according to an embodiment of the present invention.

FIG. 3 exemplarily illustrates a flowchart of a method for managing treatment of behavioral health conditions, according to an embodiment of the present invention.

FIG. 4 exemplarily illustrates a screenshot of a user inference that enable a clinician to provide score by analyzing the client response, according to an embodiment of the present invention.

FIG. 5 exemplarily illustrates a table comprising a score data, a score, a weight, and a maximum score for a revive category, a restore category, and a renew category, according to an embodiment of the present invention.

FIG. 6 exemplarily illustrates a table comprising a scoring range assigned to the questions of a housing stability category, according to an embodiment of the present invention.

FIG. 7 exemplarily illustrates a table comprising a scoring range assigned to the questions of a dosing consistency category, according to an embodiment of the present invention.

FIG. 8 exemplarily illustrates a table comprising a scoring range assigned to the questions of an illicit substance category, according to an embodiment of the present invention.

FIG. 9 exemplarily illustrates a table comprising a scoring range assigned to the questions of a counseling consistency category, according to an embodiment of the present invention.

FIG. 10 exemplarily illustrates a table comprising a scoring range assigned to the questions of a physical and mental health category, according to an embodiment of the present invention.

FIG. 11 exemplarily illustrates a table comprising a scoring range assigned to the questions of a legal issue category, according to an embodiment of the present invention.

FIG. 12 exemplarily illustrates a table comprising the questions of a renew gap category, according to an embodiment of the present invention.

FIG. 13 exemplarily illustrates a table comprising a scoring range assigned to the questions of an interpersonal stability category, according to an embodiment of the present invention.

FIG. 14 exemplarily illustrates a table comprising a scoring range assigned to the questions of a medication management category, according to an embodiment of the present invention.

FIG. 15 exemplarily illustrates a screenshot of a user interface displaying a dashboard comprising a score data, and a graphical representation of score data, according to an embodiment of the present invention.

### DETAILED DESCRIPTION OF EXAMPLE EMBODIMENTS

A description of embodiments of the present innovation will now be given with reference to the Figures. It is expected that the present innovation may be embodied in other specific forms without departing from its spirit or essential characteristics. The described embodiments are to be considered in all respects only as illustrative and not restrictive.

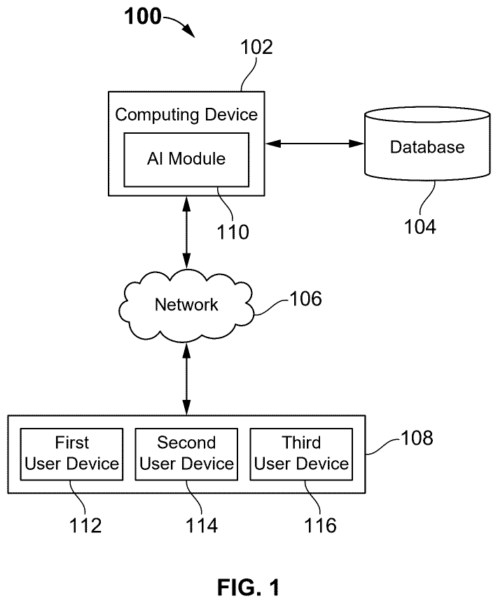

FIG. 1 exemplarily illustrates an environment 100 of a system for managing treatment of behavioral health conditions including opioid and substance use disorder, according to an embodiment of the present invention. This system is designed to extend beyond the application in opioid and substance use disorder treatment, allowing integration into mental health, behavioral health, and co-occurring disorder environments. The system provides a diagnostic-agnostic foundation that dynamically adapts to the clinical condition through a structured scoring system, AI augmentation, workflow usability, and documentation quality. The system comprises at least one computing device 102, at least one database 104 in communication with the computing device 102. The system further comprises one or more user devices 108 associated with a user. The user devices 108 and the database 104 are in communication with the computing device 102 via a network 106.

The user device 108 includes at least one first user device 112 associated with a client or a patient, at least one second user device 114 associated with a clinician, and at least one third user device 116 associated with a representative of an organization. The user device 108 is configured to provide an interface to access the services provided by the computing device 102. The interface, for example, an application that allows the user device 108 to wirelessly connect and access the computing device 102 via the network 106. The interface could be a web-based application. The user device 108 includes, but not limited to, a desktop computer, a laptop computer, a mobile phone, a personal digital assistant, and the like.

The network 106 generally represents one or more interconnected networks configured to enable communication between the user devices 108, the computing device 102 and the database 104. The network 106 may include packet-based wide area networks (such as the Internet), local area networks (LAN), private networks, wireless networks, satellite networks, cellular networks, paging networks, and the like. A person skilled in the art will recognize that the network 106 may also be a combination of more than one type of network. For example, the network 106 may be a combination of a LAN and the Internet. In addition, the network 106 may be implemented as a wired network or a wireless network or a combination thereof.

The database 104 is in communication with the computing device 102. In an example, the database 104 resides in the computing device 102. In another example, the database 104 resides separately from the computing device 102. Regardless of the location, the database 104 comprises a memory to store and organize data for use by the computing device 102. The database 104 comprises a client data, a clinician data, a score data including a category score data and a recovery score data, and a progress data. The progress data includes score data of each client assessment.

The computing device 102 is at least one of a server, a general-purpose computer, a special-purpose computer, a workstation, a desktop, a laptop, a tablet, a mobile phone, a mainframe, a supercomputer and a server farm. Although the computing device 102 is illustrated as a single device, the functions performed by the computing device 102 could be performed using any suitable number of computing devices 102. The computing device 102 comprises one or more artificial intelligence (AI) module 110. The AI module 110 comprises one or more Large Language Models (LLM). The Large Language Models (LLM) is fine-tuned with one or more training data sources. The training data source includes a specific data relevant to SUD treatment, guidelines and policies from Federal Guidelines, State Regulations and industry recognized Evidence-Based Practices (EBP).

The computing device 102 is configured to provide a questionnaire comprising the plurality of categories. Each category comprises the set of questions to assess the progress of the client. The category comprises housing stability category, dosing consistency category, illicit substance category, counseling consistency category, physical and mental health category, legal issues impact category, medication management category, interpersonal relationship category and renew gap category. The clinician could ask follow up questions for each category to improve accuracy of score. The computing device 102 is configured to use a combination of questions of one or more categories to assess the recovery of the client.

The computing device 102 is further configured to enable the clinician to input a score for each question of each category based on analyzing response of client to each question.

The computing device 102 is configured to apply predefined scoring guidelines for the input score and generate the score data. The score data includes the category score for each category and the recovery score for the questionnaire. The computing device 102 is further configured to assign weighted scores to each question based on a relative impact of each question on the recovery success of the client to generate the score data.

The computing device 102 is configured to provide a dashboard-comprising a score data, and a graphical representation of score data for the individual clients and for a group of clients. The computing device 102 is further configured to provide a graphical representation of progress data for individual clients and for the group of clients.

The computing device 102 enables to assess performance of different organization by assessing recovery of group of clients of different organization using the score data.

The computing device 102 is configured to enable the clinician to continuously evaluate and track the progress of the client in treatment by using the score data and the questionnaire for the regular period of time until the recovery score reaches a predefined score. The computing device 102 is further configured to enable the clinician to update the score for each category of questions after each client assessment. The score data of each client assessment is included in the progress data. The computing device 102 is configured to enable the clinician to provide the treatment plan based on current score data and progress data of the client.

The computing device 102 is configured to enable the client to access the treatment plan provided by the clinician. The computing device 102 is configured to analyze the progress data, and identify trends. The computing device 102 is further configured to provide optimized treatment recommendations to update the treatment plan based on score data and progress data of the client.

The computing device 102 is configured to share the progress data of clients across clinicians and organizations. In one embodiment, the progress data is shared across organizations, counselors and agencies. The computing device 102 enable to assess the overall recovery health of the entire census of clients. The computing device 102 enables users, for example, policy makers and regulators, to compare the performance of organizations. In one embodiment, the computing device 102 is configured to measure performance across clinicians, and evaluate treatment plan characteristics across the client. The computing device 102 is in communication with one or more external systems. The external system comprises an electronic health record (EHR) system and a uniform data system (UDS). The electronic health records (EHR) systems are accessed via a secure application programming interface (APIs), for example, Methisoft. The electronic health record (EHR) system is configured to ensure real-time synchronization of the client data across multiple organizations. Further, the EHR supports streamlined workflows and ensures that the data used for assessment is always current.

The uniform data system (UDS) is a data warehouse that stores the standardized reporting data from firms conducting a toxicology test for opioid treatment programs (OTPs).

The system enables to calculate and deliver the SUD mortality risk index. Further, the de-identified toxicology screens of client are integrated to the system that enables to predict SUD mortality risk. Further, the system enables to predict SUD mortality risk through the combination of the client responses to the questionnaire of each category and several test levels from toxicology screens. The computing 102 created quantitative relationships between the client responses to predict SUD mortality risk. Further, the scores are assigned for each of the clients in the clinic. The system maintains the input of current and previous clients that experienced fatal overdoses. Further, the current and previous data served helps to predict mortality.

The computing device 102 is configured to determine the client improvement by measuring and evaluating the progress of the client based on client performance. The computing device 102 is configured to update the recovery score for the client and provides recognition or reward for client progress. The computing device 102 is further configured to facilitate relapse detection, relapse monitoring, and emotional monitoring by analyzing the score data, client responses, and historical progress data to identify patterns indicative of relapse risk and emotional distress. The computing device 102 is configured to connect with wearable devices associated with patient and collect biometric data of the patient. The biometric data is mapped to the questionnaire to enable real-time recalibration of the score data and AI-driven decision support. The computing device 102 is configured to maintain real-time consistency of scoring indices, clinical documentation, and treatment planning across multi-agency, multi-county behavioral health networks. The computing device 102 is further configured to generate personalized recovery trajectories by analyzing temporal patterns in a multi-category scoring index and adjusting clinical plans through an AI-enhanced feedback engine. The computing device 102 is configured to provide role-specific interfaces to deliver AI-guided documentation prompts, compliance tracking, and personalized analytics tailored to counselors, medical staff, and administrative users.

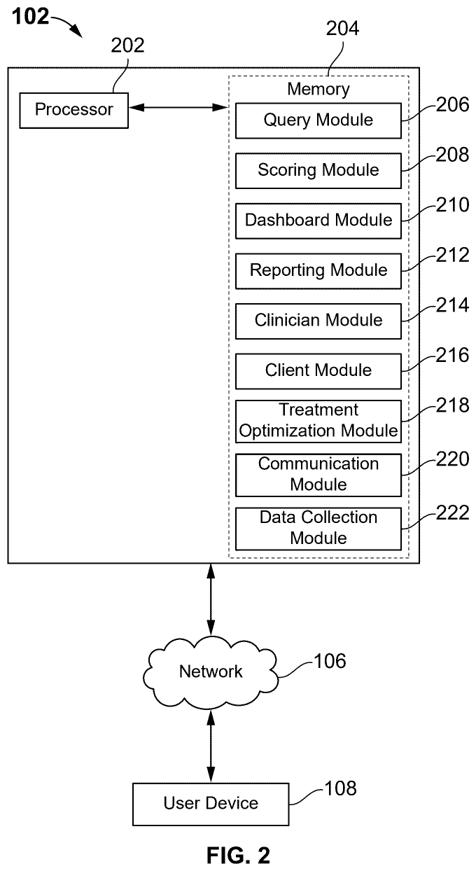

FIG. 2 exemplarily illustrates a block diagram 200 of the computing device 102, according to an embodiment of the present invention. The computing device 102 comprises one or more processors 202 and one or more memories 204. The computing device 102 further comprises one or more artificial intelligence (AI) modules 110. The processor 202 is configured to execute one or more program modules. The program modules comprise a query module 206, a scoring module 208, a dashboard module 210, a reporting module 212, a clinician module 214, a client module 216, a treatment optimization module 218, a communication module 220 and a data collection module 222.

The query module 206 is configured to provide a questionnaire comprising the plurality of categories. Each category comprises the set of questions to assess progress of the client. The query module 206 is further configured to enable the clinician to input a score for each question of each category based on analyzing response of client to each question. The clinician could ask follow up questions to improve accuracy of score.

The scoring module 208 is configured to apply predefined scoring guidelines for the input score. The scoring module 208 is further configured to generate the score data including the category score data for each category and the recovery score data for the questionnaire. The scoring module 208 is further configured to assign weighted scores to each question. The weighted scores are assigned based on the relative impact of each question on recovery success of the client to generate the score data.

The data collection module 222 is configured to connect with wearable devices associated with patient and collect biometric data of the patient. The biometric data is mapped to the questionnaire to enable real-time recalibration of the score data and AI-driven decision support. The system supports the ingestion of biometric and behavioral data from wearable devices, which dynamically influence scoring categories. For example, heart rate irregularities may affect physical/mental health category, and activity and sleep disruptions may influence interpersonal functioning, medication management category. These inputs enhance real-time patient engagement modeling and relapse prediction, distinct from prior systems that do not tightly couple biometric signals with structured, explainable index scoring.

The dashboard module 210 is configured to provide a dashboard comprising a score data, and a graphical representation of score data for the individual clients and for a group of clients. The dashboard module 210 is further configured to provide a graphical representation of progress data for individual clients and for the group of clients. The reporting module 212 enables to assess performance of different organizations by assessing recovery of group of clients of different organization using the score data.

The clinician module 214 is configured to enable the clinician to continuously evaluate and track the progress of the client in treatment. The clinicians evaluate and track by using the score data and the questionnaire for the regular period of time until the recovery score reaches a predefined score. The clinician module 214 is further configured to enable the clinician to update the score for each category of questions after each client assessment. The clinician module 214 is configured to enable the clinician to provide the treatment plan based on current score data and progress data of the client.

The client module 216 is configured to enable the client to access the treatment plan provided by the clinician. The treatment optimization module 218 is configured to analyze the progress data, identify trends, and provide optimized treatment recommendations to update the treatment plan based on score data and progress data of the client. The communication module 220 is configured to share the progress data of clients across clinicians and organizations.

The system is designed for federated deployment, allowing seamless synchronization of treatment data, scores, and clinical documentation across multiple clinics and agencies, EHR systems (via FHIR/HL7), and counties or jurisdictions. The framework of the system provides portable continuity of care and supports public-private integration across fragmented behavioral health systems. The system provides a decentralized, interoperable treatment management system that maintains real-time consistency of scoring indices, clinical documentation, and treatment planning across multi-agency, multi-county behavioral health networks. The system introduces longitudinal modeling of each patient's treatment journey using scores over time. This results in individualized “recovery trajectories,” visualized through score heatmaps, stability risk projections, and time-based compliance flags.

This temporal intelligence allows for adaptive treatment plan recalibration, co-authored by both clinician and patient, and supported by AI-derived clinical coaching insights. The system generates personalized recovery trajectories by analyzing temporal patterns in a multi-category scoring index and adjusting clinical plans through an AI-enhanced feedback engine.

The system features distinct user interfaces and AI-driven prompts tailored to specific clinical roles. The user interface enables counselors to receive real-time guidance on subjective, objective, assessment, and plan (SOAP) notes accuracy, completeness, and compliance. The user interface enables clinicians and medical staff to access medication management summaries generated from dosing consistency and substance test results. The user interface enables supervisors to utilize performance dashboards that track scoring distributions and documentation compliance across staff. This role-sensitive user experience ensures that functionality aligns with regulatory, operational, and clinical needs, distinguishing it from generic AI documentation systems.

The system is configured to uniquely combine a quantitative index framework and qualitative enhancement layer, AI-supported treatment personalization over time, real-time compliance monitoring, multi-jurisdictional interoperability, and biometric-enhanced predictive analytics.

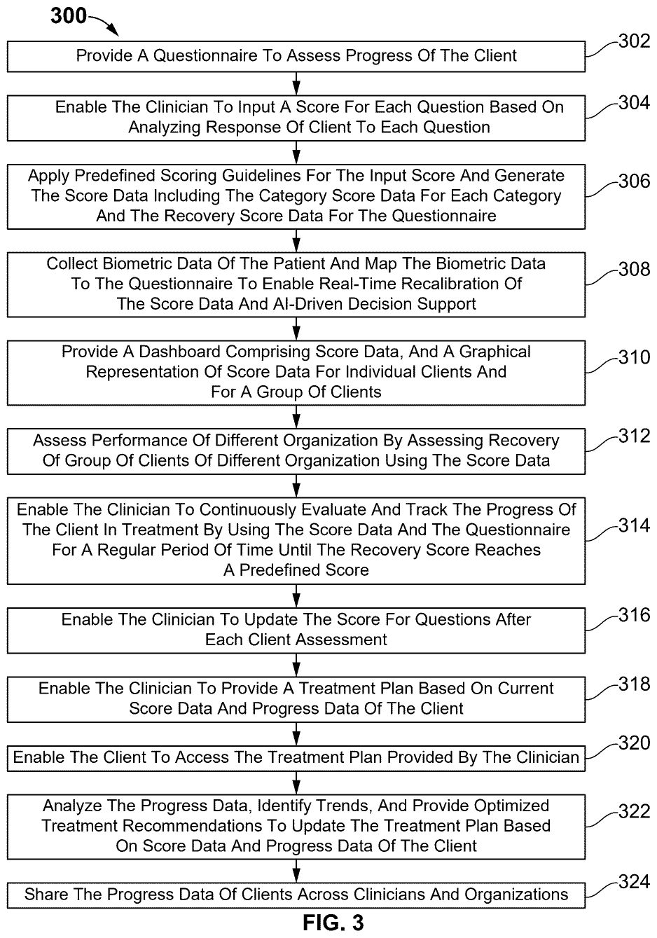

FIG. 3 exemplarily illustrates a flowchart 300 of a method for managing treatment of behavioral health conditions, according to an embodiment of the present invention. The method is executed in the system comprising at least one computing device 102, at least one database 104 in communication with the computing device 102. The computing device 102 comprises one or more artificial intelligence (AI) module 110. The database 104 comprises the client data, the clinician data, the score data including category score and recovery score, a progress data including score data of each client assessment. The system further comprises at least one user device 108 associated with a user. The user device 108 and the database 104 are in communication with the computing device 102 via the network 106.

At step 302, the query module 206, at the computing device 102, is configured to provide the questionnaire to assess progress of the client. The questionnaire comprising the plurality of categories. Each category comprises the set of questions to assess progress of the client.

At step 304, the query module 206, at the computing device 102, is configured to enable the clinician to input a score for each question based on analyzing response of client to each question.

At step 306, the scoring module 208, at the computing device 102, is configured to apply predefined scoring guidelines for the input score and generate the score data including the category score data for each category and the recovery score data for the questionnaire. At step 308, the data collection module 222, at the computing device 102, is configured to collect biometric data of the patient and map the biometric data to the questionnaire to enable real-time recalibration of the score data and AI-driven decision support.

At step 310, the dashboard module 210, at the computing device 102, is configured to provide the dashboard comprising score data, and the graphical representation of score data for individual clients and for the group of clients.

At step 312, the reporting module 212, at the computing device 102, is configured to assess performance of different organization by assessing recovery of group of clients of different organization using the score data.

At step 314, the clinician module 214, at the computing device 102, is configured to enable the clinician to continuously evaluate and track the progress of the client in treatment by using the score data and the questionnaire for a regular period of time until the recovery score data reaches a predefined score.

At step 316, the clinician module 214, at the computing device 102, enable the clinician to update the score data for each category of questions after each client assessment.

At step 318, the clinician module 214, at the computing device 102, is configured to enable the clinician to provide a treatment plan based on current score data and progress data of the client.

At step 320, the client module 216, at the computing device 102, is configured to enable the client to access the treatment plan provided by the clinician.

At step 322, the treatment optimization module 218, at the computing device 102, is configured to analyze the progress data, identify trends. Further, the treatment optimization module 218 provides optimized treatment recommendations to update the treatment plan based on score data and progress data of the client.

At step 324, the communication module 220, at the computing device 102, enable to share the progress data of clients across clinicians and organizations. The computing device 102 is configured to connect with wearable devices associated with patient and collect biometric data of the patient. The biometric data is mapped to the questionnaire to enable real-time recalibration of the score data and AI-driven decision support. The computing device 102 is configured to maintain real-time consistency of scoring indices, clinical documentation, and treatment planning across multi-agency, multi-county behavioral health networks. The computing device 102 is further configured to generate personalized recovery trajectories by analyzing temporal patterns in a multi-category scoring index and adjusting clinical plans through an AI-enhanced feedback engine. The computing device 102 is configured to provide role-specific interfaces to deliver AI-guided documentation prompts, compliance tracking, and personalized analytics tailored to counselors, medical staff, and administrative users.

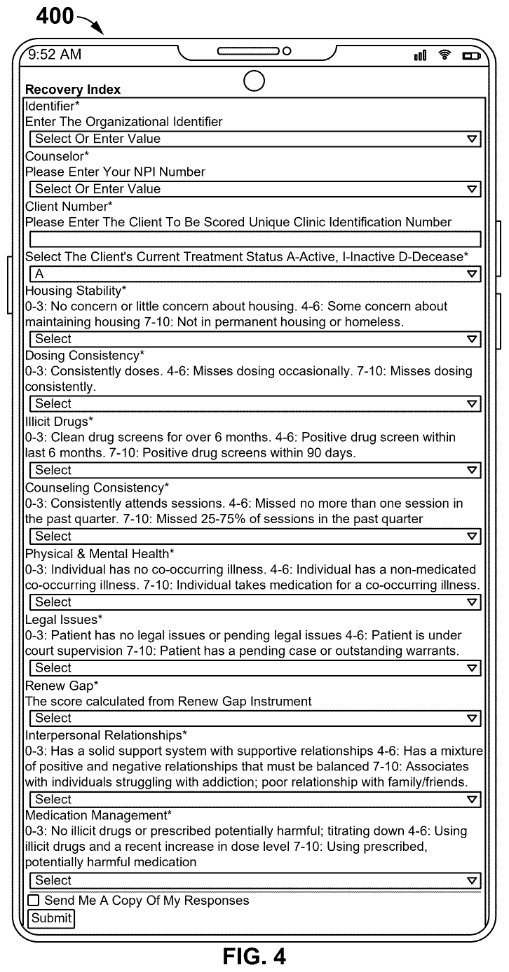

FIG. 4 exemplarily illustrates a screenshot 400 of the user interface that enables the clinician to provide the score by analyzing the client response, according to an embodiment of the present invention. The user interface displays one or more options that enable the clinician to provide input score for each question of each category based on analyzing the response of the client. The user interface allows the clinician to select an identifier, a counselor, a client number, and a client current treatment status related to the client. The user interface is configured to provide one or more options to indicate the current treatment status. For example, the options include active, inactive, and deceased. The option ‘active’ indicates that the client is currently undergoing the treatment. The option ‘inactive’ indicates that the client's treatment is paused. The option ‘deceased’ indicates that the client has passed away. Further, the user interface provides a score range for different responses of the questionaries. The user interface enables the clinician to select the score depending on the response of the client.

The user interface provides a score range for responses of questions related to the housing stability category. For, example, a score range 0-3 score indicates no concern or little concern about housing, 4-6 indicates some concern about maintaining housing, and 7-10 indicates the absence of permanent housing or a state of homelessness. The clinician could select the score depending on the response of the client to the questions related to the housing stability category.

The user interface provides a score range for response of questions related to the dosing consistency category. For, example, a score range 0-3 indicates the client is consistent in taking doses, 4-6 indicates occasional the client missed doses, and 7-10 indicates that the client frequently missed doses. The clinician could select the score depending on the response of the client to the questions related to the dosing consistency category.

The user interface provides a score range for response of questions related to the illicit drugs category. For, example, a score range 0-3 indicates the clients who have clean drug screening for over six months, 4-6 indicates the client with a positive drug screening within the last six months, and 7-10 indicates the client with a positive screening within the last three months. The clinician could select the score depending on the response of the client to the questions related to the illicit drugs category.

The user interface provides a score range for response of questions related to the counseling consistency category. For, example, a score range 0-3 indicates that the client consistently attended sessions, 4-6 indicates that the client missed more than one session in the past quarter, and 7-10 indicates that the client attended only 25%-75% of sessions in the past quarter. The clinician could select the score depending on the response of the client to the questions related to the counseling consistency category.

The user interface provides a score range for response of questions related to the physical and mental health category. For, example, a score range 0-3 indicates that the client does not have a co-occurring illness, 4-6 indicates that the client having a non-medicated co-occurring illness, and 7-10 indicates that the client having a medicated co-occurring illness. The clinician could select the score depending on the response of the client to the questions related to the physical and mental health category.

The user interface provides a score range for response of questions related to the legal issues category. For, example, a score range 0-3 indicates that the client does not have pending legal issues, 4-6 indicates that the client is under court supervision, and 7-10 indicates that the client has pending cases or outstanding warrants. The clinician could select the score depending on the response of the client to the questions related to the legal issues category.

The user interface provides a score range for response of questions related to the renew gap category. The clinician could select the score depending on the score is calculated from the renew gap Instrument. The user interface provides a score range for response of questions related to the interpersonal relationship category. For, example, a score range 0-3 indicates that the client having a strong and supportive relationships with family and friends, 4-6 indicates that the client having a balance of positive and negative relationships, and 7-10 indicates that the client is struggling with addiction and has poor relationships with family and friends. The clinician could select the score depending on the response of the client to the questions related to the interpersonal relationship category.

The user interface provides a score range for response of questions related to the medication management category. For, example, a score range 0-3 indicates that the client does not use of illicit or harmful substances, 4-6 indicates that the client uses illicit substance with increase dosage, and 7-10 indicates that the client uses prescribed and potentially harmful medications. The clinician could select the score depending on the response of the client to the questions related to the medication management category. Further, the user interface provides a ‘submit’ option to summit the form. The clinician could click the ‘submit’ option to submit the form.

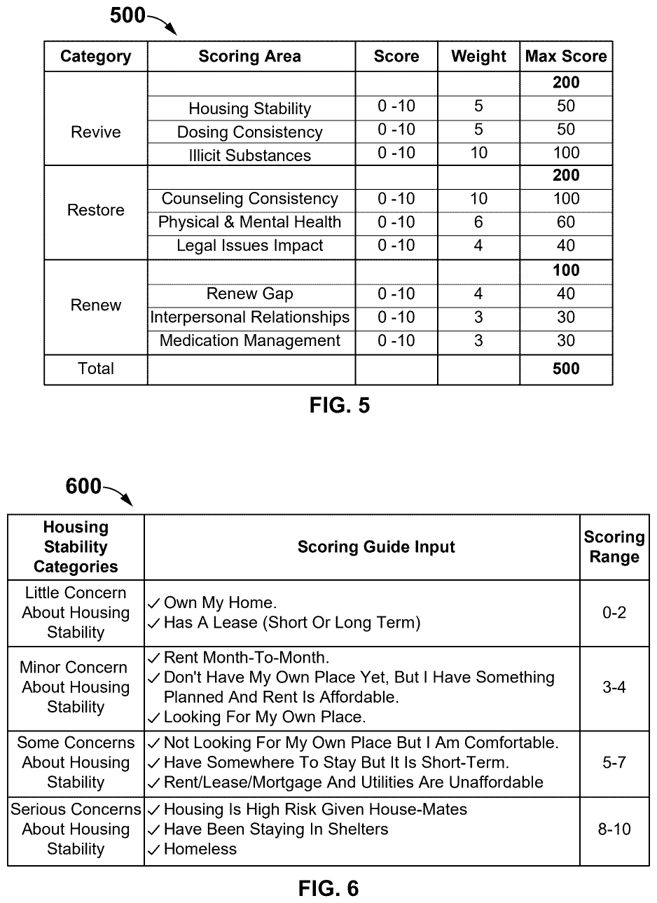

FIG. 5 exemplarily illustrates a table 500 comprising a scoring area, a score, a weight, and a maximum score for different categories of the questionnaire, according to an embodiment of the present invention. The categories including the housing stability category, the dosing consistency category, and the illicit substance category are grouped under the revive category.

The categories including the counseling consistency category, the physical and mental health category, and the legal issues impact category are grouped under the restore category.

The categories including the renew gap category, the interpersonal relationship category, and the medication management category are grouped under the renew category. The score refers to the input score provided by the clinician. The maximum score refers to the category score generated for each category. The clinician assigns the score by analyzing the response of the client to the questionaries helps to assess the client's recovery. The system provides a configurable recovery evaluation and documentation support system utilizing a three-category scoring index of revive, restore, and renew, designed for substance use disorder, mental health, and behavioral health conditions, with AI-enhanced adaptation for treatment plans and documentation workflows. Further, the biometric data from wearable devices is mapped to revive, restore, and renew variables to enable real-time recalibration of treatment scoring and AI-driven decision support.

For example, the score ranges for each questionary of the three categories ranges in the scale of zero to ten, the maximum score assigned to the revive category is 200, the maximum score assigned to the restore category is 200, and the maximum score assigned to the renew category is 100 and the maximum score for the questionnaire including all category is 500. For example, the weighted scores for housing stability category, dosing consistency category, and illicit substances category are 5,5, and 10, respectively. The weighted scores for counseling consistency category, physical and mental health category, and legal issue impact category are 10, 6, and 4, respectively. The weighted scores for medication management category, interpersonal relationships category and renew gap category is 3, 3, and 4, respectively.

In an example, the housing stability category is evaluated based on the client response to the questions of the housing stability status. For example, the questions related to the housing stability status include, but not limited to, does client currently own a house, duration of current housing, risk of losing house, unstable housing situation, housing affordability, safety, and access to stable living conditions. The dosing consistency category is evaluated based on the client response to the questions of the dosing consistency category. For example, the question related to dosing consistency category includes the number of doses missed by the client in the past 30 days. For example, the illicit substance category is evaluated based on the on the duration of the client avoiding the usage of illicit substance.

In an example, the counseling consistency category is evaluated based on the number of sessions missed in the past three months by the client. In an example, the physical and mental health category is evaluated based on the management of the physical and mental health of client with or without the use of medications. In an example, the legal issues impact category is evaluated based on the client response to the questions of the legal matters.

In an example, the renew category is evaluated based on queries related to financial, resources, independence, hobbies, well-being, health, and support from family and friends. In an example, the interpersonal relationship category is evaluated based on the relationship between the client and the other people including family or friends, and ability of the client to set boundaries with family and friends. In an example, the medication management category is evaluated based on the duration that the client has adhered to continuous treatment and remained free from illicit drug use.

FIG. 6 exemplarily illustrates a table 600 comprising the scoring range assigned to the questions of the housing stability category, according to an embodiment of the present invention. The system enables to evaluate housing stability of the client based on predefined categories, corresponding scoring guide inputs, and assigned score ranges. The housing stability category is evaluated based on level of concern related to housing stability. For example, the housing stability category classifies levels of concern regarding housing stability into four tiers including little concern, minor concern, some concern, and serious concern.

The individuals categorized under little concern about housing stability receive a score range of 0-2. The individuals categorized under little concern include individuals who own home or have a lease agreement for either a short or long-term duration. The individuals classified under minor concern about housing stability receive a score range of 3-4, which includes individuals who rent housing on a month-to-month basis, do not currently have stable housing but have an affordable plan in place, or are actively searching for housing. The individuals classified under some concern about housing stability receive a score range of 5-7. This category includes individuals who is not seeking for new housing but feel comfortable in the current living situation, those residing in short-term housing arrangements, and those who find rental or mortgage costs unaffordable. The individuals classified under serious concern about housing stability receive a score range of 8-10. This category includes individuals living in high-risk housing situations, such as cohabitation with unsafe housemates, reliance on shelters, or experiencing homelessness. The scoring process is implemented via the user device 108, which allows a clinician to input a score based on a client's responses to specific queries regarding the housing stability. These queries include, but not limited to: “Do you own or rent your home?”, “Do you live with other people in the home?”, “Who do you live with?”, “Do you contribute to bills in the household?”, “Are you at risk of losing your housing?”, and “Are you experiencing homelessness?”. Based on the responses, the clinician assigns an appropriate score within the corresponding range to accurately reflect the individual's housing stability status.

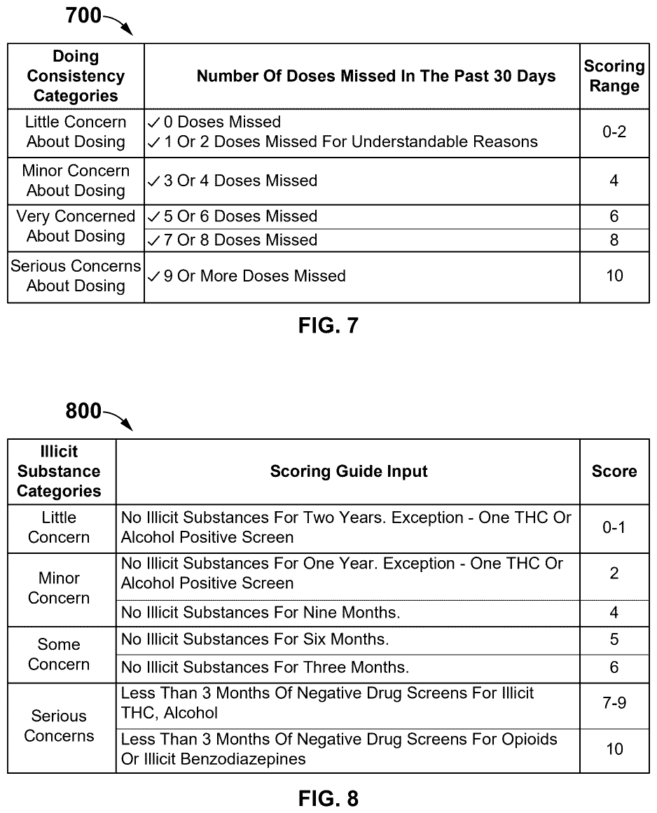

FIG. 7 exemplarily illustrates a table 700 comprises the scoring range assigned to the questions of the dosing consistency category, according to an embodiment of the present invention. The system enables to evaluate the dosing consistency of the client based on predefined categories, corresponding scoring guide inputs, and assigned score ranges. The dosing consistency category is evaluated based on levels of concern related to the dosing. For example, the dosing consistency category classifies levels of concern regarding dosing consistency into four groups including little concern, minor concern, very concern, and serious concern.

The individuals or clients categorized under little concern about dosing consistency receive a score range of 0-2. The individuals categorized under little concern include individuals who have missed 0 to 2 doses in the past 30 days for understandable reasons. The individuals classified under minor concern about dosing consistency receive a score of 4. The individuals classified under minor concern includes the individuals who have missed 3 or 4 doses in the past 30 days. The individuals classified under very concern about dosing consistency receive a score range of 6-8. The individuals classified under very concern includes the individuals who have missed 5 or 6 doses in the past 30 days and receive a score of 6. The individual who missed 7 or 8 doses in the past 30 days and receive a score of 8. The individuals classified under serious concern about dosing consistency receive a score of 10. The individuals classified under serious concern includes individuals who have missed 9 or more doses in the past 30 days.

The scoring process is implemented via the user device 108, which allows the clinician to input the score based on the client's responses to specific queries regarding dosing consistency category. The scores of 3, 5, 7, and 9 are also used for the matching number of doses missed. Further, the clinician discusses the absences of client to assess any barriers that the client experiencing to adhere to the dosing schedule. Based on the responses to the queries, the clinician assigns an appropriate score within the corresponding range to accurately reflect the individual's dosing consistency status.

FIG. 8 exemplarily illustrates a table 800 comprising the scoring range assigned to the questions of the illicit substances category, according to an embodiment of the present invention. The system evaluates the illicit substance use of the client based on predefined levels of concern, corresponding scoring guide inputs, and assigned score ranges. The illicit substances category is evaluated based on levels of concern related to illicit substances use. For example, the illicit substances category classifies levels of concern regarding illicit substances into four groups including little concern, minor concern, some concern, and serious concern.

The individuals categorized under little concern about illicit substances receive a score range of 0-1. The individuals categorized under little concern include the individuals who have not used illicit substances for at least two years, except for a single positive screen for tetrahydrocannabinol (THC) or alcohol. The individuals categorized under minor concern about illicit substances receive a score range of 2-4. The individual categorized under minor concern includes individuals who have not used illicit substances for at least one year, except for one positive screen for THC or alcohol and receive a score of 2. The individual categorized under minor concern includes individuals who have not used illicit substances for nine months and receive a score of 4.

The individuals categorized under some concern about illicit substances category and receive a score range of 5-6. The individual categorized under some concern includes the individuals who have not used illicit substances for six months and receive a score of 5. The individual categorized under some concern includes the individuals who have not used illicit substances for three months and receive a score of 6. The individuals categorized under serious concern about illicit substances receive a score range of 7-10. The individual categorized under serious concern includes the individuals with less than three months of negative drug screens for illicit substances, including THC and alcohol and receive a score of 7-9. The individual categorized under serious concern includes the individuals with less than three months of negative drug screens for illicit substances such as opioids or illicit benzodiazepines and receive a score of 10. Based on the responses to the queries, the clinician assigns an appropriate score within the corresponding range to accurately reflect the individual's illicit substances use status.

The scoring process is implemented via the user device 108, which allows the clinicians to analyze the client's urine drug screen (UDS) history and current take-home phase. The clinicians discuss the duration of consecutive negative UDS results with the client. Additionally, a client's chart is reviewed to confirm any reported controlled substance prescriptions or medical marijuana cards that might qualify the UDS as negative.

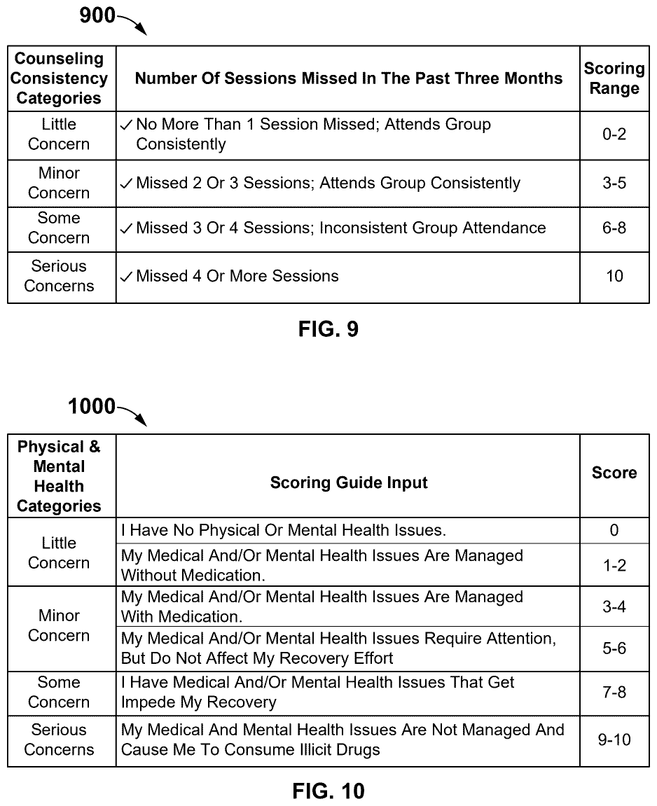

FIG. 9 exemplarily illustrates a table 900 comprising the scoring range assigned to the questions of the counseling consistency category, according to an embodiment of the present invention. The system enables to evaluate the counseling consistency of the client based on predefined levels of concern, corresponding scoring guide inputs, and assigned score ranges. The counseling consistency category is evaluated based on levels of concern related to counseling sessions. For example, the counseling consistency category classifies levels of concern regarding counseling sessions into four groups including little concern, minor concern, some concern, and serious concern.

The individuals categorized under little concern about counseling consistency receive a score range of 0-2. The individuals categorized under little concern include the individuals who have missed one session in the past three months and attended group sessions consistently. The individuals classified under minor concern about counseling consistency receive a score range of 3-5. The individuals classified under minor concern includes the individuals who have missed two or three sessions but attended group sessions regularly.

The individuals classified under some concern about counseling consistency receive a score range of 6-8. The individuals classified under some concern includes clients who have missed three or four sessions within the past three months and demonstrate inconsistent attendance at group sessions. The individuals classified under serious concern about counseling consistency receive a score of 10. The individuals classified under serious concern includes the individuals who have missed four or more sessions in the past three months.

The scoring process is implemented via the user device 108, which enables the clinician to input the score based on an analysis of the client's attendance of the sessions over the past 90 days. The clinician reviews the client's chart and scheduling history to determine the number of absences in the past 90 days. Additionally, the clinicians ask clients with specific questions to assess barriers to engagement or reasons for avoidance of counseling sessions. Based on the responses to the queries, the clinician assigns an appropriate score within the corresponding range to accurately reflect the individual's counseling consistency.

FIG. 10 exemplarily illustrates a table 1000 comprising the scoring range assigned to the questions of the physical and mental health category, according to an embodiment of the present invention. The system enables to evaluate the physical and mental health of the client based on predefined levels of concern, corresponding scoring guide inputs, and assigned score ranges. The physical and mental health category is evaluated based on levels of concern related to the physical and mental health. For example, the physical and mental health category classifies levels of concern regarding to physical and mental health are classified into four groups including little concern, minor concern, some concern, and serious concern.

The individuals categorized under little concern about physical and mental health receive a score of 0-2. The individuals categorized under little concern include the individual who does not have physical or mental health issues receives a score of 0. The individuals categorized under little concern include the individual who could manage the medical or mental health without medication receives a score range of 1-2. The individuals classified under minor concern about physical and mental health receive a score range of 3-6. The individuals classified under minor concern includes the individuals who could manage the medical or mental health conditions with medication and receive a score range of 3-4. Further, the individuals classified under minor concern includes the individuals whose medical or mental health conditions require attention but do not affect the recovery efforts receive a score range of 5-6.

The individuals classified under some concern about physical and mental health receive a score range of 7-8. The individuals classified under some concern includes the individual whose medical or mental health issues impede the recovery process. The individual categorized under serious concern about physical and mental health receive a score range of 9-10. The individual classified under the serious concern includes the individual who could not manage the medical or mental health issues and leads to the use of illicit substances.

The scoring process is implemented via the user device 108, which enables the clinician to input the score based on the client's responses to specific queries regarding the health status. The queries include, but not limited to: “Do you have any physical or mental health diagnoses?”, “Do you take any prescribed medications?”, “Do you have any symptoms that affect your ability to maintain sobriety?”, and “Do you see a doctor, psychiatrist, or any other specialist?” Based on the responses, the clinician assigns an appropriate score within the corresponding range to accurately reflect the individual's physical and mental health status. Based on the responses to the queries, the clinician assigns an appropriate score within the corresponding range to accurately reflect the individual's physical and mental health status.

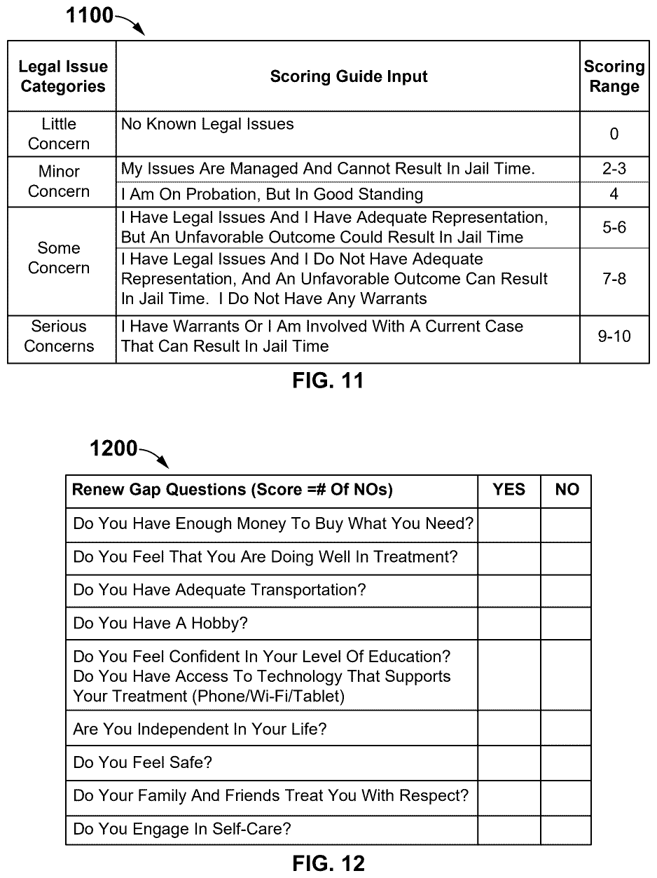

FIG. 11 exemplarily illustrates a table 1100 comprising the scoring range assigned to the questions of the legal issues impact category, according to an embodiment of the present invention. The system enables to evaluate the legal issues impact on the client based on predefined levels of concern, corresponding scoring guide inputs, and assigned score ranges. The legal issues impact category is evaluated based on levels of concern related to the client's current legal status. For example, the legal issues impact category classifies levels of concern regarding the legal issues impact into four groups including little concern, minor concern, some concern, and serious concern.

The individuals categorized under little concern about legal issues impact receive a score of 0. The individual categorized under little concern includes the individual who does not have known legal issues. The individuals classified under minor concern about legal issues impact receive a score range of 2-4. The individuals classified under minor concern includes the individual who could manage the legal issues and do not have a risk of jail time receives a score range of 2-3. The individuals classified under minor concern includes the individual who are on probation but remain in good standing receives a score of 4.

The individuals classified under some concern about legal issues receive a score range of 5-8. The individuals classified under some concern includes the individual with legal issues who have adequate representation but face an outcome that could result in jail time receive a score range of 5-6. The individuals classified under some concern includes the individual with legal issues who lack the adequate representation but do not have any active warrants receive a score of 7-8.

The individuals classified under serious concern about legal issues receive a score range of 9-10. The individuals classified under serious concern includes the individual with active warrants or the individual involved in ongoing legal cases that could lead to jail time.

Based on the responses to the queries, the clinician assigns an appropriate score within the corresponding range to accurately reflect the individual's legal issues status. The scoring process is implemented via the user device 108, which allows the clinician to input a score based on the client's responses and documentation review. The clinician assigns a score after discussing the client's current legal status and examining related documents, including the legal section of the American Society of Addiction Medicine (ASAM) assessment and any release of information documents.

FIG. 12 exemplarily illustrates a table 1200 comprising the scoring range assigned to the questions of the renew gap category, according to an embodiment of the present invention. The system enables to evaluate the renew gap of the client based on predefined questions, corresponding scoring guide inputs, and assigned score ranges. The renew gap category is evaluated based on the number of questions the client answered with “No.”

The scoring process is implemented via the user device 108, which allows the clinician to input the score based on the client's responses to specific queries regarding the renew gap. The queries include, but not limited to: Do you have enough money to buy what you need? Do you feel that you are doing well in treatment? Do you have adequate transportation? Do you have a hobby? Do you feel confident in your level of education? Do you have access to technology that supports your treatment (phone/Wi-Fi/tablet) Are you independent in your life? Do you feel safe? Do your family and friends treat you with respect? Do you engage in self-care? Further, the score is assigned based on the number of questions the client answered with “No”. Based on the responses, the clinician assigns an appropriate score within the corresponding range to accurately reflect the individual's well being status. The score assigned to the renew gap category corresponds directly to the number of questions the client answered with “No.” The scoring process is implemented via the user device 108, which allows the clinician to input scores based on the client's responses.

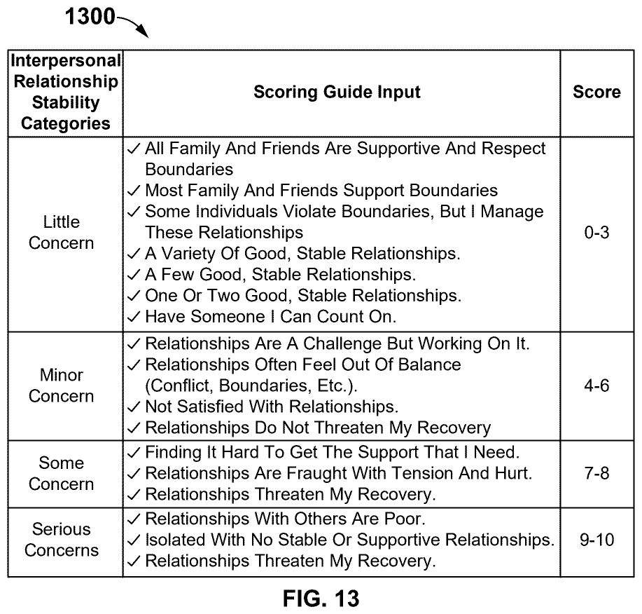

FIG. 13 exemplarily illustrates a table 1300 comprising the scoring range assigned to the questions based on the interpersonal relationship stability category, according to an embodiment of the present invention. The system enables to evaluate the interpersonal relationship stability of the individual based on predefined categories, corresponding scoring guide inputs, and assigned score ranges. The interpersonal relationship stability category is evaluated based on the level of concern related to the interpersonal relationship stability. For example, the interpersonal relationship stability category classifies levels of concern regarding the interpersonal relationship stability into four groups including little concern, minor concern, some concern, and serious concern.

The individuals categorized under little concern about interpersonal relationship stability receive a score range of 0-3. The individuals categorized under little concern include individuals who have supportive family and friends and respects the boundaries, individuals whose family and friends support the individual's boundaries, individuals who have been violate boundaries but able to manage the relationships, individuals who have a good and stable relationships, individuals who have a few good and stable relationships, individuals who have one or two strong and stable relationships, individuals who have someone whom the individuals could count on for support.

The individuals classified under minor concern about interpersonal relationship stability receive a score range of 4-6. The individuals classified under minor concern includes individuals who relationship is challenging but the individuals are working on the relationship, individuals who struggle to balanced relationships due to conflict or boundary issues, individuals who are dissatisfied of their relationships, individuals who relationships do not affect the recovery.

Individuals classified under some concern about interpersonal relationship stability receive a score range of 7-8. The individuals classified under some concern includes the individuals who struggle to receive the support they need, individuals who have relationships that are fraught tension and emotional distress, individuals who have relationships that pose a risk to the recovery.

Individuals classified under serious concern about interpersonal relationship stability receive a score range of 9-10. The individuals classified under serious concern includes the individuals who has poor relationship with others, individual who isolate themselves from supportive relationships, individual who have the relationships that threaten the recovery of the individual.

The scoring process is implemented via the user device 108, which allows the clinician to input scores based on the client's responses to specific queries. These queries include, but not limited to: which relationships are the most important to you in your life? who are you in communication with on a regular basis? do you feel that you have a strong support system? do you experience any conflict, arguments, or fights with people in your life? Based on the responses to the queries, the clinician assigns an appropriate score within the corresponding range to accurately reflect the individual's interpersonal relationship stability status.

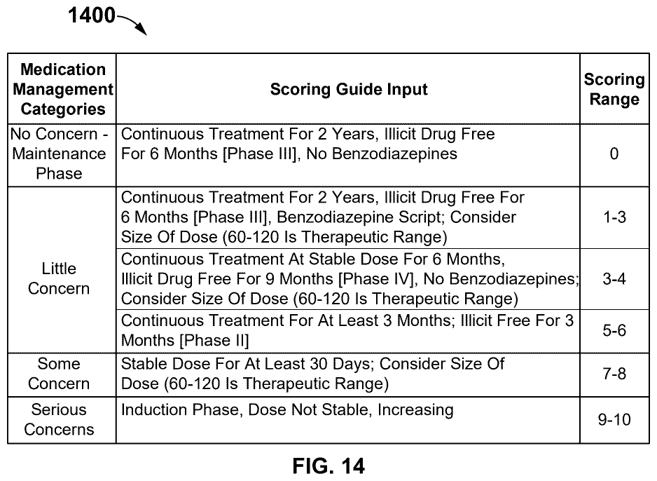

FIG. 14 exemplarily illustrates a table 1400 comprising the scoring range assigned to the questions of the medication management category, according to an embodiment of the present invention. The system enables to evaluate the medication management of the client based on predefined levels of concern, corresponding scoring guide inputs, and assigned score ranges. The medication management category is evaluated based on level of concern related to the medication management. For example, the medication management category classifies levels of concern include no concern-maintenance phase, little concern, some concern, and serious concern.

The individuals categorized under no concern-maintenance phase about medication management receive a score range of 0. The individuals categorized under no concern-maintenance phase includes the individuals who have been in continuous treatment for two years, illicit drug-free for six months in Phase III, and haven't used benzodiazepines.

The individuals categorized under little concern about medication management receive a score range of 1-6. The individuals categorized under little concern include the individuals who have been in continuous treatment for two years, illicit drug-free for six months in Phase III, but have an active benzodiazepine prescription within a therapeutic dose range of 60-120 mg receive a score range of 1-3. The individuals categorized under little concern include the individuals who have been in continuous treatment at a stable dose for six months, illicit drug-free for nine months in Phase IV, with no benzodiazepine use receive a score range of 3-4. The individuals categorized under little concern include the individuals who have been in continuous treatment for at least three months, illicit drug-free for three months in Phase II receive a score range of 5-6.

The individuals categorized under some concern about medication management receive a score range of 7-8. The individuals categorized under some concern includes the individual who have been on a stable dose for at least 30 days but require ongoing monitoring of therapeutic dose range of 60-120 mg.

The individuals categorized under serious concern about medication management receive a score range of 9-10. The individuals categorized under serious concern includes the individuals who are in the induction phase with increasing dose and do not have stable dose.

Further, the score is assigned based on the clinician analysis of the client's admission date, medical notes, and dosing history to assess the level of stability of the client regarding medication management, USD results and the presence of prescriptions. The score is assigned by analyzing the UDS results are positive for benzodiazepines, including clonazepam, alprazolam, diazepam, lorazepam, oxazepam, and temazepam.

The scoring process is implemented via the user device 108, which allows the clinician to input a score based on the client's responses to specific queries regarding the medication management. The queries include, but not limited to: are you experiencing any withdrawal symptoms? do you feel stable at your current dose? how long have you been in treatment? and when was the last time you increased your dose? Further, check to ensure the client is scheduled for a medical evaluation or has seen medical staff in accordance with facility requirements. Based on the responses to the queries, the clinician assigns an appropriate score within the corresponding range to accurately reflect the individual's medication management status.

The recovery score is determined by measuring and evaluating the progress of the client based on the client performance. The recovery score of the client is updated and provides recognition for client progress. The predictive equation for measuring the recovery score is as follows:

Recovery score˜f=a₁(Housing Stability)+a₂(Dosing Consistency)+a₃(Illicit Substances)+b₁(Counseling Consistency)+b₂(Physical & Mental Health)+b₃(Legal Issues)+c₁(Renew Gap)+c₂(Interpersonal Relationship Stability)+c₃(Medication Management)+xₙ(Toxicology Screen Results)+yᵢ(Substance Risk Factor).

It should be understood that the questions, scoring ranges, and classifications provided are merely examples and may vary based on different implementations of the system. The predefined categories, scoring guide inputs, and assigned score ranges can be modified to accommodate various assessment criteria, clinical preferences, or specific use cases.

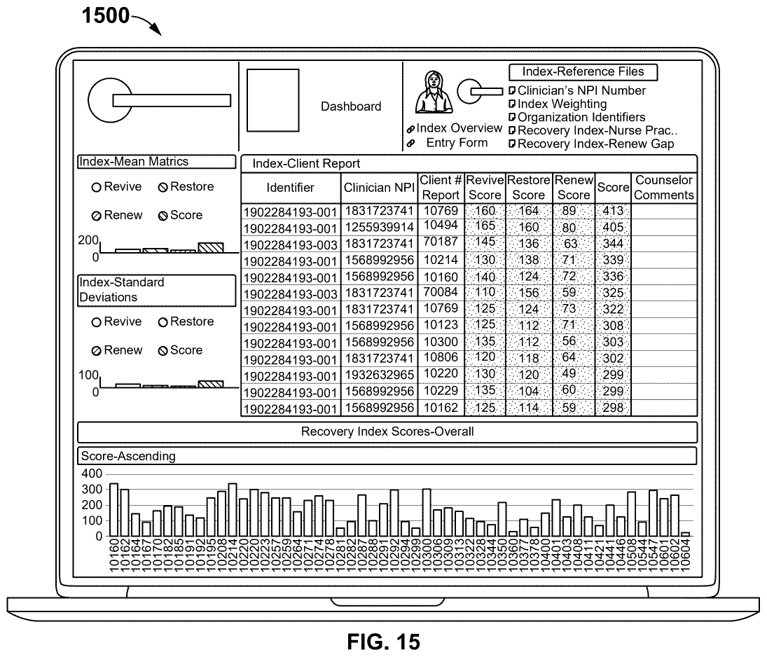

FIG. 15 exemplarily illustrates a screenshot 1500 of a user interface displaying a dashboard comprising a score data, and a graphical representation of score data, according to an embodiment of the present invention. The dashboard comprises the score data, and the graphical representation of score data for the individual clients and for the group of clients. The dashboard displays the graphical representation of progress data for individual clients and for the group of clients. The dashboard displays an index reference file. For example, the index reference file comprises a clinician's national provider identifier (NPI) number, index weighting, organization identifiers, and recovery index-renew gap. Further, the dashboard displays recovery index scores-overall and score-ascending. The dashboard further displays an index client report comprises an identifier, clinician NPI, client report, revive score, restore score, renew score, and the recovery score. For example, the identifier is 1902284193-001, the clinician NPI is 1831723741, and the client report is 10789. The individual scores are listed as revive score: 180, restore score: 184, renew score: 189, and the score is 413. The counselor comments column provides a space for notes or observations related to the client's progress or treatment. Further, the dashboard is displayed with the score assigned to the revive category, the restore category and the renew category. The index-mean metrics comprises the score data and the graph for the revive, restore, and renew category. The index-standard deviation comprises the score data and the graph for the revive, restore, and renew category.

Advantageously, the system focuses on robust technical implementation that includes structured AI integration and secure APIs for EHR to ensure real-time synchronization of client data across multiple platforms and to ensure secure data transfer mechanism. The system enables to share the client recovery and the treatment plan across the organization, agencies, and clinicians. Further, the system enables real time tracking of client's health to ensure the treatment plans for the client. Further, the system provides both quantitative data and qualitative data that helps in understanding a client's progress and treatment requirements. The quantitative data could be the scores assigned to the information.

The system ensures the accessibility for various user environments while maintaining compatibility with existing technology infrastructures in clinics and other healthcare settings. Additionally, the system is utilized for relapse detection, relapse monitoring mechanism and emotional monitoring, and effectively addressing gaps, for example structured scoring methodologies. The relapse monitoring mechanism relies on scoring methodologies including revive, restore, and renew categories. The system enables to compare the performance of different organizations based on client data. Further, the system also enables to assess and compare the performance of individual clinicians across the organization. The system enables to analyze and assess the characteristics of treatment plans across the client base. The system helps to assess the agency performance by calculating and communicating the recovery score of the client. The system enables to initiate lifesaving measures based on score and to track stabilization in critical domains. The system enables to identify a comprehensive set of elements that predict recovery. The scoring system of the present invention enables clinicians to focus on recovery, progress, results in addition to administrative compliance. Further, the system is a distinct and adaptable AI framework that could be applied across a wide range use cases, while preserving its unique treatment structure and integrated AI-enhanced architecture.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device, or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A system for managing treatment of behavioral health conditions, comprising:
one or more user devices including at least one first user device associated with a client, at least one second user device associated with a clinician, and at least one third user device associated with a representative of an organization;
at least one database comprises a client data, a clinician data, a score data including a category score data and a recovery score data, a progress data including the score data of each client assessment, and
at least one computing device in communication with at least one database and the user devices via a network, wherein the computing device comprises one or more artificial intelligence (AI) modules, wherein the computing device comprises at least one memory configured to store a set of program modules and at least one processor configured to execute one or more program modules, wherein the modules comprise:
a query module configured to provide a questionnaire comprising a plurality of categories, wherein each category comprises a set of questions to assess progress of the client, and wherein the query module is configured to enable the clinician to input a score for each question of each category based on analyzing response of client to each question,
a scoring module configured to apply predefined scoring guidelines for the input score and generate the score data including the category score data for each category and the recovery score data for the questionnaire,
a data collection module configured to connect with wearable devices associated with patient and collect biometric data of the patient, wherein the biometric data is mapped to the questionnaire to enable real-time recalibration of the score data and AI-driven decision support.
a dashboard module configured to provide a dashboard comprising score data, and a graphical representation of score data for individual clients and for a group of clients, and
a reporting module enables to assess performance of different organization by assessing recovery of group of clients of different organization using the score data.

2. The system of claim 1, wherein the modules further comprise:
a clinician module configured to enable the clinician to continuously evaluate and track the progress of the client in treatment by using the score data and the questionnaire for a regular period of time until the recovery score data reaches a predefined score, and enable the clinician to update the score data for each category of questions after each client assessment, wherein the clinician module is configured to enable the clinician to provide a treatment plan based on current score data and progress data of the client,
a client module configured to enable the client to access the treatment plan provided by the clinician,
a treatment optimization module configured to analyze the progress data, identify trends, and provide optimized treatment recommendations to update the treatment plan based on score data and progress data of the client, and
a communication module configured to share the progress data of clients across clinicians and organizations.

3. The system of claim 1, wherein the category comprises housing stability category, dosing consistency category, illicit substances category, counseling consistency category, physical and mental health category, legal issues impact category, medication management category, interpersonal relationships category and renew gap category.

4. The system of claim 1, wherein the dashboard module is further configured to provide a graphical representation of progress data for individual clients and for the group of clients.

5. The system of claim 1, wherein the scoring module is configured to assign weighted scores to each question based on a relative impact of each question on recovery success of the client to generate the score data.

6. The system of claim 1, wherein the computing device is in communication with one or more external systems including Electronic Health Record (EHR) system, and Uniform Data System, to at least one of receive and access toxicology screen results to predict SUD mortality risk and to generate score data, and wherein the computing device is further configured to facilitate relapse detection, relapse monitoring, and emotional monitoring by analyzing the score data, client responses, and historical progress data to identify patterns indicative of relapse risk and emotional distress.

7. The system of claim 1, wherein the behavioral health conditions include mental health conditions and substance use disorder.

8. The system of claim 1, wherein the computing device is configured to maintain real-time consistency of scoring indices, clinical documentation, and treatment planning across multi-agency, multi-county behavioral health networks.

9. The system of claim 1, wherein the computing device is configured to generate personalized recovery trajectories by analyzing temporal patterns in a multi-category scoring index and adjusting clinical plans through an AI-enhanced feedback engine, and wherein the computing device is configured to provide role-specific interfaces to deliver AI-guided documentation prompts, compliance tracking, and personalized analytics tailored to counselors, medical staff, and administrative users.

10. A method for managing treatment of behavioral health conditions, comprising
providing one or more user devices, a database and at least one computing device in communication with at least one database and the user devices via a network, wherein the user devices includes at least one first user device associated with a client, at least one second user device associated with a clinician, and at least one third user device associated with a representative of an organization, wherein the database comprises a client data, a clinician data, a score data including category score and recovery score, a progress data includes score data of each client assessment, wherein the computing device comprises one or more artificial intelligence (AI) modules, wherein the computing device comprises at least one memory configured to store a set of program modules and at least one processor configured to execute one or more program modules;
providing, at the computing device via a query module, a questionnaire comprising a plurality of categories, wherein each category comprises a set of questions to assess progress of the client, and enabling the clinician to input a score for each question of each category based on analyzing response of client to each question;
applying, at the computing device via a scoring module, predefined scoring guidelines for the input score and generate the score data including the category score for each category and the recovery score for the questionnaire;
providing, at the computing device via a dashboard module, a dashboard comprising score data, and a graphical representation of score data for individual clients and for a group of clients, and enabling, at the computing device via a reporting module, to assess performance of different organization by assessing recovery of group of clients of different organization using the score data.

11. The method of claim 10, further comprises a step of:
enabling, at the computing device via a clinician module, the clinician to continuously evaluate and track the progress of the client in treatment by using the score data and the questionnaire for a regular period of time until the recovery score reaches a predefined score, and enabling the clinician to update the score for each category of questions after each client assessment.

12. The method of claim 11, further comprises a step of:
enabling, at the computing device via the clinician module, the clinician to provide a treatment plan based on current score data and progress data of the client.

13. The method of claim 12, further comprises a step of:
enabling, at the computing device via a client module, the client to access the treatment plan provided by the clinician.

14. The method of claim 10, further comprises a step of:
enabling, at the computing device via a treatment optimization module, to analyze the progress data, identify trends, and provide optimized treatment recommendations to update the treatment plan based on score data and progress data of the client.

15. The method of claim 10, further comprises a step of:
enabling, at the computing device via a communication module, to share the progress data of clients across clinicians and organizations.

16. The method of claim 10, wherein the category comprises housing stability category, dosing consistency category, illicit substances category, counseling consistency category, physical and mental health category, legal issues impact category, medication management category, interpersonal relationships category and renew gap category, and wherein the behavioral health conditions include mental health conditions and substance use disorder.

17. The method of claim 10, further comprises a step of:
providing, at the computing device via the dashboard module, a graphical representation of progress data for individual clients and for the group of clients.

18. The method of claim 10, wherein the step of generating the score data involves assigning weighted scores to each question based on a relative impact of each question on recovery success of the client.

19. The method of claim 10, wherein the computing device is in communication with one or more external systems including Electronic Health Record (EHR) system, and Uniform Data System, to at least one of receive and access toxicology screen results to predict SUD mortality risk and to generate score data, and wherein the computing device is further configured to facilitate relapse detection, relapse monitoring, and emotional monitoring by analyzing the score data, client responses, and historical progress data to identify patterns indicative of relapse risk and emotional distress.

20. The method of claim 10, further comprising steps of:
connecting, via a data collection module at the computing device, with wearable devices associated with patient and collect biometric data of the patient, wherein the biometric data is mapped to the questionnaire to enable real-time recalibration of the score data and AI-driven decision support;
maintaining, at the computing device, real-time consistency of scoring indices, clinical documentation, and treatment planning across multi-agency, multi-county behavioral health networks;
generating, at the computing device, personalized recovery trajectories by analyzing temporal patterns in a multi-category scoring index and adjusting clinical plans through an AI-enhanced feedback engine, and
providing, at the computing device, role-specific interfaces to deliver AI-guided documentation prompts, compliance tracking, and personalized analytics tailored to counselors, medical staff, and administrative users.
